(19) United States
(12) Patent Application Publication
(10) Pub. No.: US 2024/0274283 A1
(43) Pub. Date: Aug. 15, 2024

(54) THERAPEUTIC GAS DELIVERY SYSTEM AND OPERATION

(71) Applicant: Mallinckrodt Pharmaceuticals Ireland Limited, Dublin (IE)

(72) Inventors: Declan Fox, Clontarf (IE); Matthew Roche, Bray (IE)

(21) Appl. No.: 18/441,180

(22) Filed: Feb. 14, 2024

Related U.S. Application Data
(60) Provisional application No. 63/445,421, filed on Feb. 14, 2023.

(51) Int. Cl. G16H 40/67

(52) U.S. Cl. CPC G16H 40/67

(57) ABSTRACT

A method is provided for managing a gas delivery device by a server. The method includes receiving a request to provision a serial number of a PCB from a manufacturing application executing at a manufacturer; providing the serial number and a test firmware to the manufacturer; providing a product firmware to the manufacturing application for installation into the gas delivery device; receiving encrypted data from a gas delivery gateway that is configured to receive performance information from the gas delivery device; decrypting the encrypted data to yield the performance information and storing the performance information in a historical repository; receiving one or more actions that are performed at a servicing application; logging each of the one or more actions in the historical repository; and providing at least one token or data to the servicing application for each of the one or more actions.

### CLAIM OF PRIORITY

This application claims the benefit of U.S. Provisional Application No. 63/445,421, filed on Feb. 14, 2023, the entire contents of which are herein incorporated by reference in its entirety.

### FIELD

The present disclosure is directed to methods of managing a gas delivery device by a server.

### BACKGROUND

Medical facilities may use therapeutic gas, such as nitric oxide (NO) gas, for administering to patients. The systems and devices that deliver the therapeutic gas often need maintenance and oversight to ensure proper functioning of these systems and devices. This is typically a labor and time intensive process. Therefore, there is a need for a therapeutic gas delivery system and operation that can improve manufacturing, maintenance, and operation of therapeutic gases such as NO and simplify administration of therapeutic gas delivery systems within medical facilities and allow medical facilities to perform their function of providing medical care.

### SUMMARY

Provided herein is a method of managing a gas delivery device by a server. The method can include receiving a request to provision a serial number of a printed circuit board (PCB) from a manufacturing application executing at a manufacturer, where the PCB is integrated into a gas delivery device, providing the serial number and a test firmware to the manufacturer, wherein the test firmware is configured to program hardware devices during manufacturing, providing a product firmware to the manufacturing application for installation into the gas delivery device, receiving encrypted data from a gas delivery gateway that is configured to receive performance information from the gas delivery device, wherein the gas delivery device is configured to transmit information to the gas delivery gateway under predetermined circumstances, decrypting the encrypted data to yield the performance information and storing the performance information of the gas delivery device in a historical repository, receiving one or more actions that are performed at a servicing application for servicing or analyzing the gas delivery device, logging each of the one or more actions in the historical repository, and providing at least one token or data to the servicing application for each of the one or more actions.

In an aspect, the method may further include receiving encrypted data from a mobile device that is configured to interface with the gas delivery device, where the gas delivery device is configured to transmit information to the gas delivery gateway under predetermined circumstances. In some aspects the method may even further include decrypting the encrypted data to yield the performance information and storing the performance information of the gas delivery device in the historical repository. The gas delivery device may be configured to identify one of the mobile device or the gas delivery gateway and provide the encrypted data based on whether gas is being delivered.

In some aspects, when the gas delivery device is delivering the gas, the gas delivery device may be configured to omit transmission of the encrypted data. In some aspects, when the gas delivery device is not in a shutdown mode and is not delivering the gas, the gas delivery device may be configured transmit of the encrypted data on a long range wireless area network. In other aspects, when the gas delivery device is in a shutdown mode, the gas delivery device may be configured transmit of the encrypted data on a long range wireless area network or a short range wireless area network.

In an aspect, the method may further include detecting at least one communication failure of the gas delivery device and providing a recommendation to service the gas delivery device.

In some aspects, the method may further include providing a manifest to the manufacturing application to install the manifest into the gas delivery device, where the manifest identifies components that can be replaced in the gas delivery device. The one or more actions may include an action to replace a first component of the gas delivery device based on an expiration date or a failure.

In an aspect, the method may further include generating an updated manifest that identifies a second component that replaces the first component, wherein the action to replace the first component is stored in the historical repository.

Further provided herein is a system for managing a gas delivery device by a server. The system can include a processor in communication with a memory. The memory can include instructions executable by the processor to receive a request to provision a serial number of a printed circuit board (PCB) from a manufacturing application executed at a manufacturer, wherein the PCB is integrated into the gas delivery device, providing the serial number and a test firmware to the manufacturer, wherein the test firmware is configured to program hardware devices during manufacturing, receive encrypted data from a gas delivery gateway that is configured to receive performance information from the gas delivery device, wherein the gas delivery device is configured to transmit information to the gas delivery gateway under predetermined circumstances, decrypt the encrypted data to yield the performance information and storing the performance information of the gas delivery device in a historical repository, receive one or more actions that are performed at a servicing application for servicing or analyzing the gas delivery device, log each of the one or more actions in the historical repository, and provide at least one token or data to the servicing application for each of the one or more actions.

In an aspect, the memory including instructions executable by the processor can be further configured to receive the encrypted data from a mobile device that is configured to interface with the gas delivery device, wherein the gas delivery device is configured to transmit information to the gas delivery gateway under predetermined circumstances, and decrypt the encrypted data to yield the performance information and storing the performance information of the gas delivery device in the historical repository.

In an aspect, the gas delivery device can be configured to identify one of the mobile device or the gas delivery gateway and provide the encrypted data based on whether gas is being delivered. In another aspect, when the gas delivery device is delivering the gas, the gas delivery device can be configured to omit transmission of the encrypted data. In another aspect, when the gas delivery device is not in a shutdown mode and is not delivering a gas, the gas delivery device is configured to transmit the encrypted data on a long-range wireless area network. In an aspect, when the gas delivery device is in a shutdown mode, the gas delivery device can be configured to transmit the encrypted data on a long-range wireless area network or a short-range wireless area network.

In an aspect, the memory including instructions executable by the processor can be further configured to detect at least one communication failure of the gas delivery device and provide a recommendation to service the gas delivery device. In an aspect, the memory including instructions executable by the processor can be further configured to provide a manifest to the manufacturing application to install the manifest into the gas delivery device, wherein the manifest identifies components that can be replaced in the gas delivery device. In an aspect, the one or more actions can comprise an action to replace a first component of the gas delivery device based on an expiration date or a failure.

In various aspects, the memory including instructions executable by the processor can be further configured to generate an updated manifest that identifies a second component that replaces the first component, wherein the action to replace the first component is stored in the historical repository.

### BRIEF DESCRIPTION OF THE DRAWINGS

Details of one or more aspects of the subject matter described in this disclosure are set forth in the accompanying drawings and the description below. However, the accompanying drawings illustrate only some typical aspects of this disclosure and are therefore not to be considered limiting of its scope. Other features, aspects, and advantages will become apparent from the description, the drawings and the claims.

FIG. 1 illustrates an operational block diagram of a nitric oxide (NO) gas delivery operation in accordance with aspects of the disclosure;

FIG. 2 illustrates a block diagram of an onsite deployment of a medical gas delivery system that is deployed in a medical facility in accordance with aspects of the disclosure;

FIG. 3A illustrates a detailed block diagram of the NO gas delivery system in accordance with aspects of the disclosure;

FIG. 3B illustrates a portion of the detailed block diagram of FIG. 3A;

FIG. 3C illustrates a portion of the detailed block diagram of FIG. 3A;

FIG. 3D illustrates a portion of the detailed block diagram of FIG. 3A;

FIG. 3E illustrates a portion of the detailed block diagram of FIG. 3A;

FIG. 4 illustrates a sequence diagram of manufacturing a main printed circuit board (PCB) of a therapeutic gas delivery device that can be employed in the therapeutic gas delivery system in accordance with aspects of the disclosure;

FIG. 5 illustrates a sequence diagram of manufacturing at least a portion of a gas delivery device that can be employed in the therapeutic gas delivery system in accordance with aspects of the disclosure;

FIG. 6 is a flowchart of an example method of a therapeutic gas operation in accordance with aspects of the disclosure; and

FIG. 7 shows an example of a system for implementing certain aspects of the present technology.

### DESCRIPTION

Medical facilities may use therapeutic gas for various purposes. One illustrative gas is nitric oxide (NO). The disclosed systems and techniques disclose a therapeutic gas delivery system and operation that can improve manufacturing, maintenance, and operation of therapeutic gases such as NO and simplify administration of therapeutic gas delivery systems within medical facilities and allow medical facilities to perform their function of providing medical care.

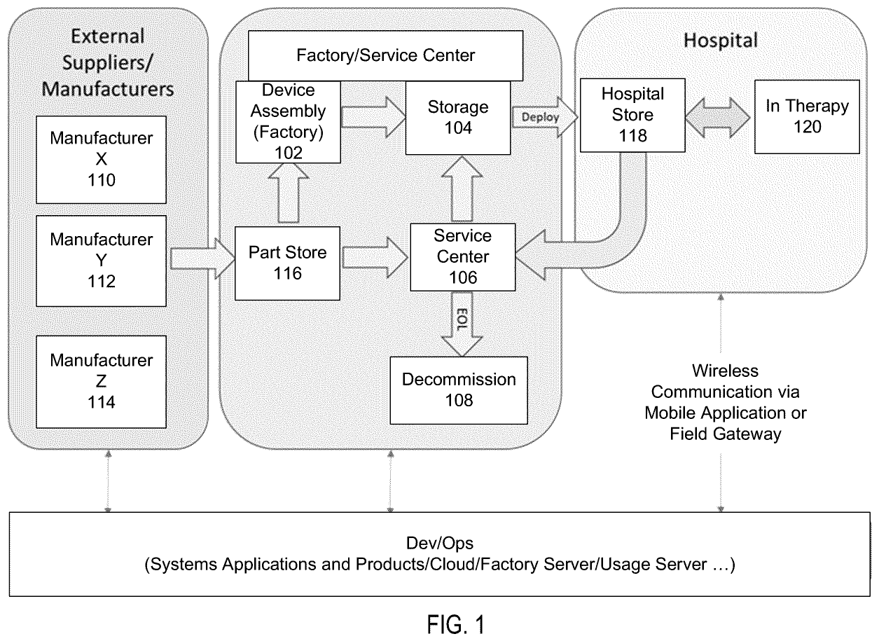

FIG. 1 illustrates an operational block diagram for manufacturing, servicing, and maintaining a therapeutic gas delivery device in accordance with aspects of the disclosure. Manufacturing a therapeutic gas delivery device includes obtaining various materials from external suppliers and manufacturers. The materials can include, for example, mechanical housings, circuit boards, tubing, wiring, amongst various other materials and components. The materials can be provided from multiple manufacturers and supplies. While FIG. 1 illustrates three manufacturers, Manufacturer X 110, Manufacturer Y 112, and Manufacturer Z 114, it will be appreciated that more or less manufacturers and external service providers can be necessary to provide the components and materials to manufacture a therapeutic gas delivery device. The therapeutic gas delivery operation includes a various suppliers and manufacturers for various materials, such as mechanical housing, circuit boards that are provided to a factory/service center that is configured to assemble the various components (or devices) used in the therapeutic gas delivery operation. An example device is a gas delivery device that is used to deliver gas to the patient for various purposes. One example of a gas is a nitric oxide (NO). For example, a gas delivery device can be used in intensive care units (ICUs), neonatal intensive care units (NICUs), pediatric intensive care units (PICUs), or other areas of a hospital to assist in the treatment of patients in need thereof. Another device of the therapeutic gas delivery operation is a gas delivery gateway that is configured to communicate with a provider of the therapeutic gas delivery operation and enable data logging, securing of any data to be compliant with various legal requirements, and other features disclosed herein.

The factory/service center can include a part store 116 that stores the various raw materials that are used to assemble the different components of the therapeutic gas delivery operation, a device assembly section 102 for assembling the various components, a storage section 104 for storing assembled and operational components of the therapeutic gas delivery operation, a service center 106 for servicing the components of the therapeutic gas delivery operation, and a decommission section 108 for decommissioning the various components. The factory/service center provides both assembly and maintenance of the components. For example, the factory/service center can be operable to manufacture a new therapeutic gas delivery device or maintain a therapeutic gas delivery device that is already in operation at a hospital. When a replacement component of the therapeutic gas delivery device is needed, the factory/service center can either send the replacement component to the hospital for self-installation, send an operator to the hospital with the replacement component to install the replacement component, or receive the therapeutic gas delivery device from the hospital and replace the component at the device assembly section 102 or the service center 106.

At the part store 116, parts supplied from external vendors and manufacturers are catalogued, invoiced, and uniquely labeled. At the device assembly section 102, the device is assembled from a collection of parts from the parts store 116. When the device is assembled, the device is assigned a unique device identifier (UDI) which identifies the device throughout its lifetime. While components of the device can be replaced during the lifetime of the device, the UDI does not change. The components used in the device are recorded throughout the lifetime of the device, and updated if replaced, maintaining a full historical record of each part which a specific device has ever contained. These historical records can provide information regarding the quality of parts received from manufacturers, the history of the devices usage rate and lifetime of certain components within a specific device which can be used to determine maintenance times, and other valuable information regarding a specific device.

The storage section 104 stores inventory of components of the therapeutic gas delivery operation such as the gas delivery devices and gas delivery gateways, and also receives components that are refreshed from the service center for distribution. In some aspects, the factory/service center can be used to distribute new/restored components to a client such as a hospital. The hospital can store unused components such as a gas delivery devices or components (filter assemblies, sensor assemblies, circuit boards, sample lines, tubing, or other components necessary for the proper operation of the therapeutic gas delivery devices) until necessary. When needed, the hospital can activate a gas delivery device for delivering gas to a patient. The gas delivery device is “in therapy” 120 when moved into a medical delivery area such as an ICU. The gas delivery devices can have a standby state in which gas may not be delivered to the patient and may be configured to deliver gas to a patient. In some aspects, the hospital can also include a hospital store 118 for storing the therapeutic gas delivery device until it is needed. Various states of the gas delivery devices are further described below.

During assembly at the device assembly section 102, a series of steps are taken to provision the device for use in the future. For example, device firmware is uploaded to the device, and device-specific information is retrieved from the device for use in the future. Some of these steps can happen at the device level and/or component level. Provisioning the device for use is described further herein.

Each of the parties (e.g., external suppliers and manufacturers of various components used in the therapeutic gas delivery operation), the factory/service center, and the client (e.g., the hospital) may be configured to connect to a dev/ops system, which is combination of development and IT operations to provide continuous delivery, maintenance, and monitoring with high software quality and hardware components. In some aspects, the dev/ops system includes a central server to manage the various devices and coordinate with various systems such as a storage system, an enterprise resource planning (ERP) system, authentication systems, and so forth.

While the device is located in the factory or service center, it can be considered to be in a “secure” environment and is permitted to accept connections from service and/or manufacturing applications. Once the device is in the “field” (e.g., hospital) it can only transmit data (e.g., communicate outwardly, meaning be the instigator of all communications) under certain defined conditions and timeframes.

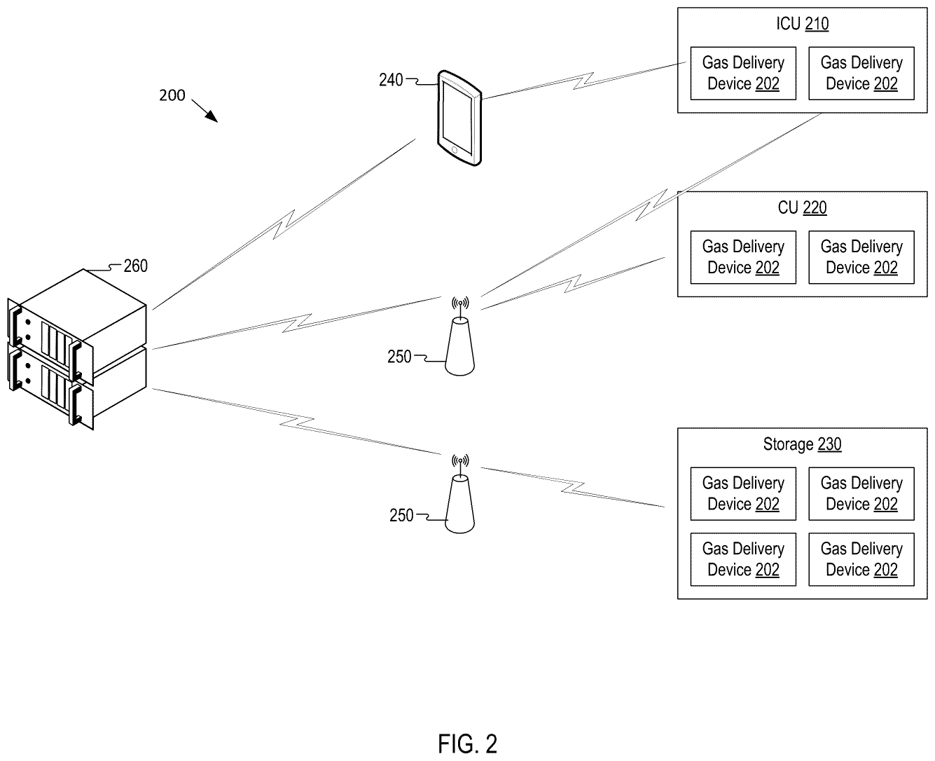

FIG. 2 illustrates a block diagram of an onsite deployment of a gas delivery system that is deployed in a medical facility in accordance with aspects of the disclosure. In particular, the system 200 includes a medical facility that has different areas to which one or more gas delivery devices 202 may be deployed or stored. For example, the medical facility includes an ICU 210, a caring unit (CU) 220, and a storage 230. In this case, the storage 230 stores the components that are not in use. The medical facility may also include an electronic device 240 (e.g., a mobile device such as a tablet, a mobile phone, etc.), and a gas delivery gateway 250. As illustrated in FIG. 2, there may be more than one gas delivery gateway 250.

In some aspects, the electronic device 240, and the gas delivery gateway 250 may be configured to receive data logged by the one or more gas delivery gateways 250. In some cases, the medical facility may have a single gas delivery gateway 250, which communicates with the electronic device 240 and/or the dev/ops system 260.

During the time that a gas delivery device 202 is deployed to a medical facility, it remains in contact with the dev/ops system 260 by way of wireless transmissions, on both a periodic and therapy-based basis. These communications can be either via a gas delivery gateway 250 or via local wireless transmissions initiated by a field agent, onsite at the medical facility, using an application on an electronic device 240 (e.g., a mobile phone, a tablet, etc.) to collect the data from the gas delivery device 202.

The gas delivery device 202 is removed from the medical facility and brought back to a factory/service center when it is deemed or reported to be faulty, or after a defined time has elapsed—periodic maintenance. During this service it is checked, components replaced if faulty or (nearing) end-of-life (e.g. sensors, batteries etc.). Potentially new product firmware is loaded, if required, and all historic data is retrieved (typically log files) and deleted from the gas delivery device 202 so that it can be re-deployed to a different hospital from where it was retrieved.

Overarching the entire lifecycle of the gas delivery device 202 is the dev/ops system 260, where the business logic, ecosystem monitoring, device management, medical/customer management software stacks exist. All constituent parts of the gas delivery device 202, the gas delivery device 202 themselves, the service history, deployment history and operational history of the gas delivery device 202 are all tracked and manipulated at the dev/ops system 260.

In some aspects, the gas delivery device 202 is configured to transmit data to the electronic device 240 during the transitions between periods of “standby” and periods of “therapy”. While in therapy, (e.g., involved in the treatment of a patient, either actively delivering NO or ready to do so), the gas delivery devices 202 may remain in this state for days or even weeks, before being returned to the “standby” state where it is typically removed from the vicinity of the patient(s) requiring treatment and may be cleaned and put into a defined storage area, in anticipation of future use.

The gas delivery device 202 is permitted to perform wireless transmissions under certain circumstances, such during defined time-bounded windows, and when the gas delivery device 202 is not actively delivering NO to a patient. The gas delivery device 202 may transmit on a long-range wireless access network (Lora WAN) and a short-range wireless network (e.g., Bluetooth Low Energy BLE) while the gas delivery device 202 is in standby state. When the gas delivery device 202 is in therapy, but not actively delivering NO, the gas delivery device 202 is permitted to use LoRaWAN again subject to transmission window limits. While the gas delivery device 202 is delivering NO, the gas delivery device 202 may not transmit at all, on any protocol.

Another procedure exists where a gas delivery device 202 is installed in a medical facility which does not have any co-located gateways such as the electronic device 240, or the gas delivery device 202 is unable to communicate autonomously with an electronic device 240 as intended. In this scenario a person visits the hospital with an electronic device 240 running a corresponding application and can collect any outstanding data transmissions from gas delivery devices 202 which are in standby using BLE as a mechanism to trigger transmission to the electronic device 240 over Wi-Fi.

In some aspects, there may be multiple co-located gas delivery gateways 250 and the gas delivery devices 202 may be configured to ensure that data is provided a single time. Various aspects can be implemented to ensure that the data is successfully retrieved by the electronic device 240 or the gas delivery gateway 250.

The gas delivery gateway 250 may be deployed to each hospital alongside the gas delivery devices 202 to collect information and act as a router for transmissions from the gas delivery devices 202, collecting the transmissions over various communication techniques such as LoRaWAN and/or Wi-Fi. The data is gathered and sent to a server for decryption and verification. In some aspects, the gas delivery gateway 250 may use a cellular (3G/2G) uplink from the gas delivery gateway 250 to the server.

If there are multiple gas delivery gateways 250 in the medical facility, for example due to transmission/reception issues according to the construction and/or layout of the hospital—each gas delivery gateway 250 will be configured similarly to collect the data from every gas delivery device 202 in that medical facility. The gas delivery devices 202 will repeatedly attempt to send unsent data (within its transmission criteria, and on the permitted radio(s)) until the first gas delivery gateway 250 has acknowledged receipt of it, at which point the gas delivery device 202 considers the data as sent and no longer attempts to send it again.

In the scenario where either there is no gas delivery gateway 250 deployed, the gas delivery gateway 250 has failed, or the gas delivery device 202 is not within range of gas delivery gateway 250 at any point in time, the un-transmitted data remains on the gas delivery device 202 until collected (and acknowledged) by an electronic device 240 running a corresponding, communicating using, for example, BLE/Wi-Fi. In some cases, a representative (agent/employee) will visit the medical facility, locate the gas delivery device 202 from which the server has not received any transmissions over an extended period of time, and will initiate a data transfer from the gas delivery device 202. The application on the electronic device 240 will then upload the received data to the server when it can do so, according to network or cellular capabilities.

The application on the electronic device 240 can also retrieve data consolidated at the gas delivery gateway 250, in the event that the gas delivery device 202 has been transmitting successful to the gas delivery gateway 250 but the uplink from the gas delivery gateway 250 to the server is not working. This could save the representative from having to search the hospital for a gas delivery device 202, as they may possibly be located in many different floors or departments.

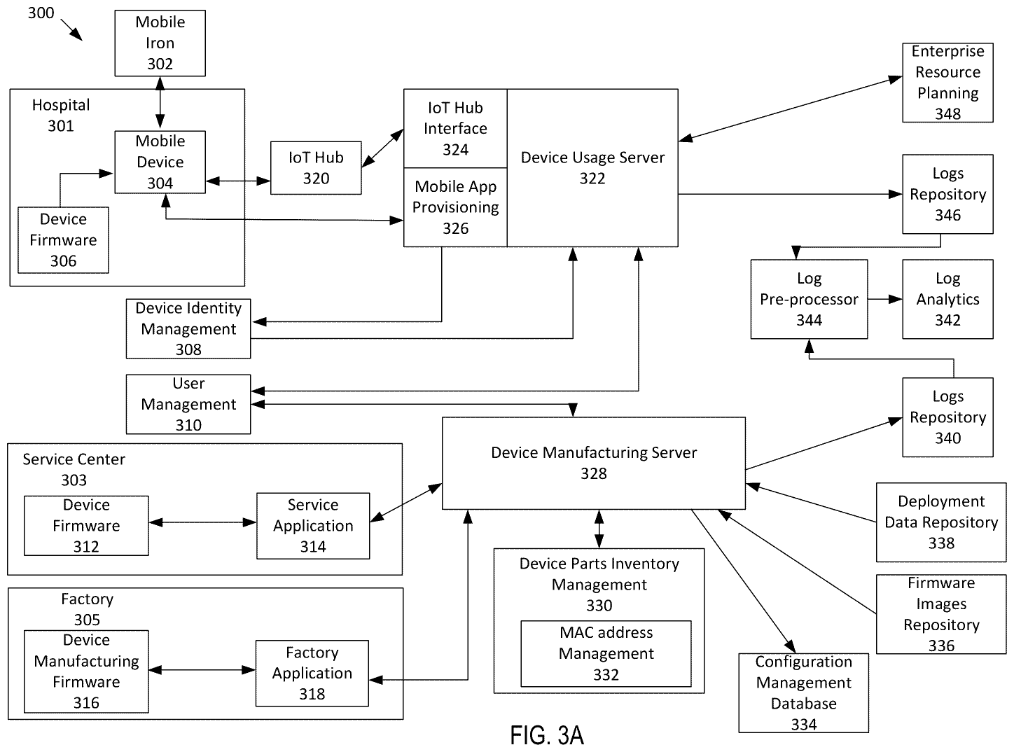

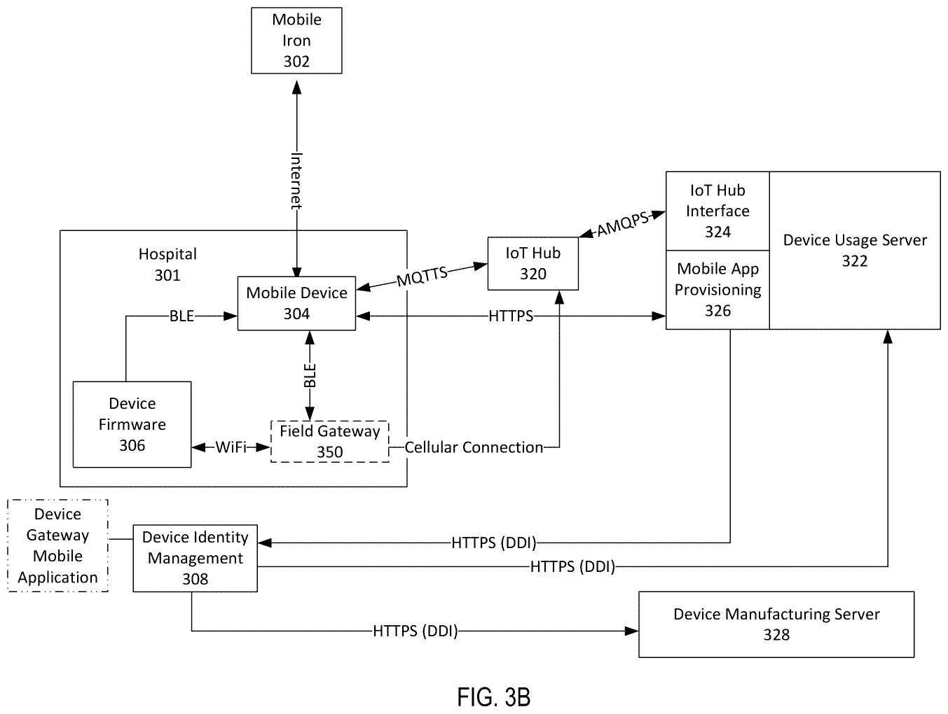

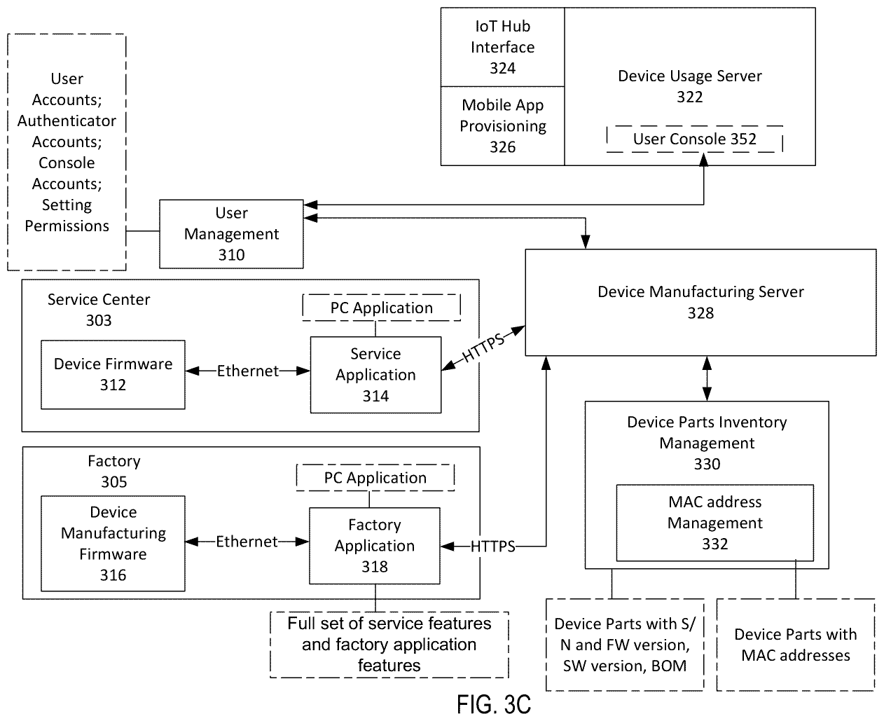

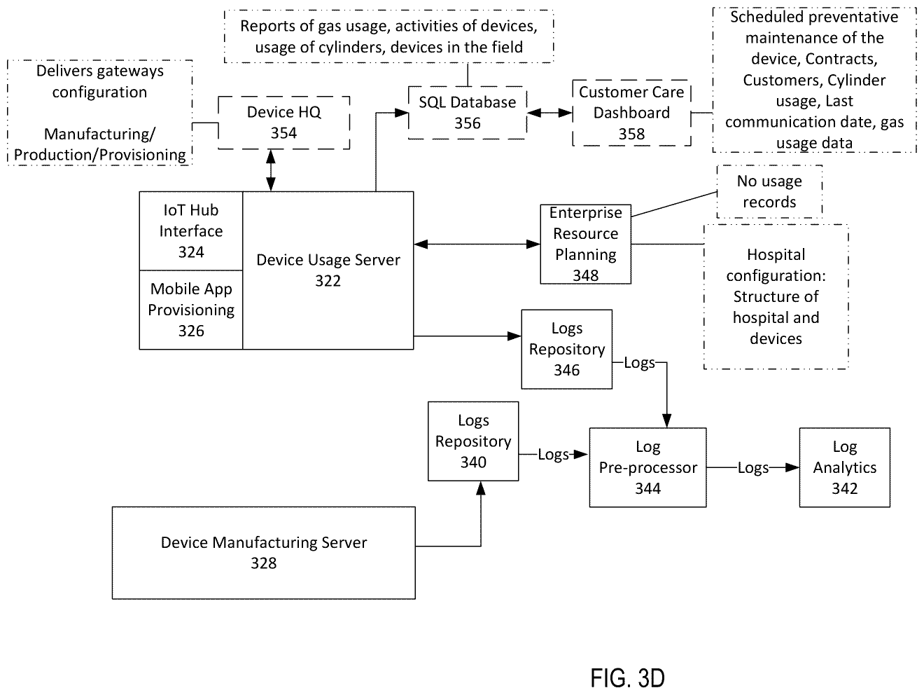

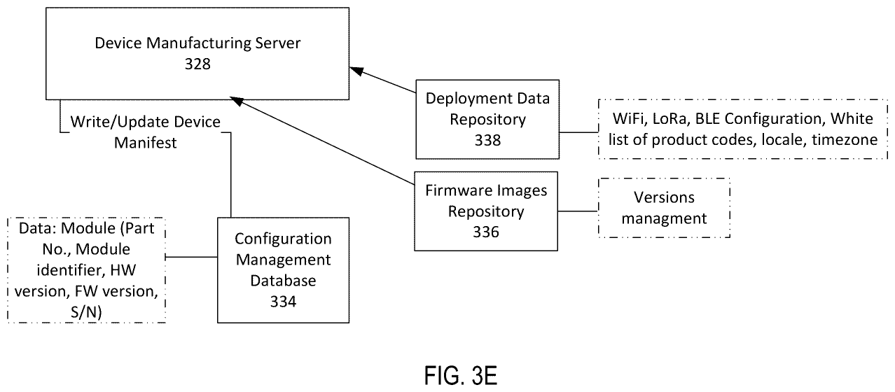

FIG. 3A illustrates a detailed block diagram of the dev/ops system 300 in accordance with aspects of the disclosure. In particular, FIG. 3A illustrates the various components necessary to implement the security, data storage, data retrieval, and other functions described herein. For example, the dev/ops system 300 can include various communication points for sending and receiving data. The dev/ops system 300 groups all of the functionality involved in the management and manufacturing of the therapeutic gas delivery devices which are deployed to hospitals including the processes of device manufacturing, service, collation of data transmissions from hospitals and generation of bills.

The device usage server 322 and the device manufacturing server 328, collectively the device server, can be operable to record parts and components which are delivered after manufacture, prior to being assembled into a device. The device server can further be operable to track which specific components are in, and generate a manifest for, each device, during manufacturing and service operations. The device server can further be operable to provide configuration data to be placed onto each device, during manufacturing and service operations. The device server can further be operable to populate field gateways with deployment configuration data to enable field gateways to receive data from devices deployed to their location. The device server can further be operable to receive transmitted data from devices in the field, decrypt the data, and verify the data's validity and integrity. The device server can further be operable to export expiry and activation dates of user-changeable components in devices (e.g., gas sensor module). The device server can further be operable to identify which specific devices or entire hospitals appear to not be transmitting data regularly or reliably. The device server can further be operable to ensure all interactions and operations performed on the devices by service or factory procedures are logged and tracked. The device server can further be operable to generate data required to facilitate the collection of records from devices over BLE/Wi-Fi using a mobile application.

The device server can have interfaces to many external components and resources including systems applications and products (SAP) (hospital data, device deployment, and billing generation), a logs repository, a factory application, a service center application, configuration management database (e.g., Azure SQL), a mobile iron (mobile device application management), a firmware image repository (location of images for software, upgrade packs, etc.), and an IoT interface (Azure IoTHub) for ingress of wireless transmissions from devices and provisioning of deployment to field gateway and mobile application.

The central components of the dev/ops system 300 are the device usage server 322 and the device manufacturing server 328. The device usage server 322 can include an IoT hub interface 324 and mobile app provisioning module 326. The device usage server 322 can be in communication with various modules, as illustrated in FIG. 3A. For example, the device usage server 322 can be in communication with a device in a hospital 301. The device firmware 306 can be configured to transmit data to an electronic device (mobile device 304) and the mobile device can transmit the data through an IoT hub 320 to the IoT hub interface 324. The mobile device 304 can also be in communication with a mobile iron 302. A device identity management module 308 can be in communication with the mobile app provisioning module 326 and the device usage server 322. The device identify management module 308 can be configured to verify the identity of a mobile device 304 thereby ensuring security. A user management module 310 can be configured to communicate with the device usage server 322 and the device manufacturing server 328.

The service center 303 can be in communication with the device manufacturing server via device firmware 312 and a service application 314. The service application allows the firmware to be uploaded at device firmware 312 as well as transmit the firmware data to the device manufacturing server 328. This can provide various information to the device usage server 322 via the user management module 310 to ensure that all parts in the device are appropriately tracked and monitored.

The factory 305 can be in communication with the device manufacturing server 328 via device manufacturing firmware 316 and a factory application 318. The factory application 318 can allows firmware to be uploaded at the manufacturing firmware 316 during device manufacture and transmit the firmware data to the device manufacturing server 328. This can provide various information to the device usage server via the user management module 310 to ensure that all parts in the device are appropriately tracked and monitored.

The device usage server can be in communication with an enterprise resource planning module 348. The enterprise research planning module 348 can be configured to store hospital configuration data such as the structure of the hospital and devices.

The device manufacturing server 328 can also be in communication with a device parts inventory management module 330. The device parts inventory management module 330 can include a MAC address management module 332. The device parts inventory module 330 can be configured to store data related to the various components in inventory to be used in the devices during servicing or manufacturing.

The device manufacturing server 328 can also be in communication with a configuration management database 334 (Azure SQL). The configuration management database 334 can be configured to securely track the identity of various components used in the manufacturing and service of the device as well as manage the components.

The dev/ops system 300 can further include a deployment data repository 338 and a firmware images repository 336 in communication with the device manufacturing server 328. The deployment data repository 338 can store and transmit data for configuring a device for deployment in a hospital. The firmware images repository 336 can be configured to store and transmit data related to the version of firmware on various devices.

The dev/ops system 300 can further include a log analytics module 342. The log analytics module 342 can be configured to determine the proper function of a device or specific components within a device. The logs analytics module 342 can receive data from the device manufacturing server 328 including data related to the components in the device via a logs repository 340. The logs analytics module 342 can also receive data related to device usage from the device usage server 32 via a logs repository 346. In some examples, the data received at the logs analytics module 342 is pre-processed by a log pre-processor module 344 (e.g., to de-encrypt the data or perform other functions) before being received at the logs analytics module 342.

FIG. 3B illustrates a detailed diagram of a portion of the dev/ops system 300. In some examples, the device firmware 306 in the hospital 301 can communicate via BLE to the mobile device 304. In another example, the device firmware 306 can communicate with a field gateway 350 via WiFi. The mobile device 304 can communicate via MQTTS to an IoT hub 320 which communicates via AMQPS to an IoT hub interface 324 of the device usage server, thereby providing usage data from the device to the device usage server 322. In another example, the device firmware 306 can communicate with the field gateway 350 which sends data to the IoT hub 320 via a cellular connection and thereby the IoT hub interface 324 and the device usage server. The mobile device 304 can be connected to the mobile iron 302 via internet.

The device identity management module 308 can be in communication with both the device manufacturing server 328 and the device usage server 322 via HTTPS (DDI). The device identity management module 308 can be a device gateway mobile application.

FIG. 3C illustrates a detailed diagram of a portion of the dev/ops system 300. In the service center 303 the device firmware can be in communication with the service application 314 via ethernet. The service application 314 can be a PC application. The service application 314 can transmit data to the device manufacturing server 328 via HTTPS. In the factory 305, the device manufacturing firmware 316 can be in communication with the factory application 318 via ethernet. The factory application 318 can be a PC application. The factory application 318 can transmit data to the device manufacturing server 328 via HTTPS. The factory application 318 can include a full set of service and factory application features.

The user management module 310 can include user accounts, authenticator accounts, console accounts, and setting permissions. The user management module 310 can be in communication with both the device usage server 322 and the device manufacturing server 328. In some examples, the device usage server can include a user console 352 to communicate with the user management module 310.

The device manufacturing server 328 can be in communication with the device parts inventory management module 330 which can include a MAC address management module 332. The device parts inventory management module 330 can include data including device parts serial numbers, firmware versions, software versions, and bills of materials. The MAC address module 332 can include MAC addresses for various device parts.

FIG. 3D illustrates a detailed diagram of a portion of the dev/ops system 300. In some examples, the device usage server 322 can be in communication with a device HQ 354, a SQL databased 356, and a customer care dashboard 358. The device HQ 354 can be configured to deliver gateway configurations to the dev/ops system and monitor and control manufacturing, production, and provisioning of devices and components. The SQL database 356 can be configured to report gas usage, activities of devices, usage of cylinders (gas cylinders used with the devices), and devices in the field. The customer care dashboard 358 can be configured to provide scheduled preventative maintenance of the device, contracts, customers, cylinder usage, last communication dates by the device, and gas usage data.

The enterprise resource planning module 348 can provide hospital configurations, structures, and structures of the devices to the device usage server 322. In some examples, the enterprise resource planning module 348 can be prevented from receiving usage data.

FIG. 3E illustrates a detailed diagram of a portion of the dev/ops system 300. The device configuration management database 334 can be written and/or updated (based on whether it is a new device manufacture or a service of a used device). The configuration management database 334 can store data device (module) data including part numbers, device (module) identifiers, hardware, firmware version, and serial number for each device and each component.

The deployment data repository 338 can store information such as the hospital communication structure (WiFi, LoRa, BLE), a white list of product codes, the locale in a hospital, and a time zone in the hospital. The deployment data repository 338 can store this data for a plurality of different hospitals where the devices are used. The firmware images repository 336 can store information for version management on various devices and device components.

The device can consist of several PCBs and assemblies, which can be manufactured by multiple suppliers. Each part can undergo post-manufacturing testing and can be assigned a unique serial number which identifies the part for the duration of its existence. Certain components can require provisioning and/or retrievel of unique data or information which are used in other parts of the lifecycle. For example, NO usage boards can have their physical identifiers (e.g., Wi-Fi and BLE MAC address, LoRaWAN DEVEUI) collated and stored alongside the unique serial number in order to provide a look-up capability mapping device identity to these fields. The dev/ops system 300 can record these serial numbers and attributes when the parts are received at the factory.

During device assembly, a set of parts and components are assembled together to create the device. At this stage the device is given a UDI which will be unchanged throughout the lifetime of the device. The provisioning of unique data (e.g., encryption keys) occurs during this step. Encryption keys are created and supplied by the dev/ops system, which also securely stores the encryption keys for future use. The device manifest is loaded onto the device, which allows the device to verify the boards which are installed in the device are the correct ones, and that the integrity of the device is assured.

The configuration files which configure the radios for wireless transmission in a hospital are also loaded, so that, the deployment of a deice to a hospital does not require further interaction. This configuration includes LoRaWAN credentials, Wi-Fi access points to which to join, and credentials for the same, Wi-Fi bands, and BLE configuration settings. The product firmware is installed, replacing any manufacturing software which was installed on the individual boards during the manufacturing process.

When devices are deployed to a hospital, the devices are not preconfigured of preassigned to a specific hospital until they are delivered to a specific hospital. Any device can potentially be delivered to any hospital. Occasionally, it may be necessary to provide additional configuration or customization to devices in certain hospitals in certain circumstances. Specific configurations for different geographical regions can include a default language, a time offset, and a list of permitted cylinder codes.

When the device is delivered to a hospital, its UDI and/or serial number is recorded as having been delivered to a hospital and this information is recorded in the dev/ops system 300. The recorded information is used to allow the device server to facilitate wireless transmission from the devices in the hospital to the dev/ops system 300.

While in the hospital, the device transitions between periods of standby and periods of therapy. The device may remain in therapy (i.e., involved in treatment of a patient) for days or even weeks, either actively delivering therapeutic gas or ready to do so. The device is permitted to perform wireless transmissions under certain circumstances, during defined time-bound windows, and only when the device is not actively delivering therapeutic gas to a patient. The device can transmit on Wi-Fi or LoRaWAN and act as a BLE server while it is in the standby state. When the device is in therapy, but not actively delivering therapeutic gas, it is permitted to use LoRaWAN only, subject to the transmission window limits. While the device is delivering therapeutic gas, it cannot transmit at all on any radio protocol.

A second procedure exists where a device is installed in a hospital which does not have any field gateways or the device is unable to communicate autonomously with a field gateway. In this scenario an operator visits the hospital with a mobile device running a mobile application and can collect any outstanding transmissions from devices which are in standby mode using BLE as a mechanism to trigger transmission to the mobile device over Wi-Fi.

The device must be collected from the hospital periodically and undergo preventative maintenance, according to a schedule at a regional service center. There can also be occasions outside of the maintenance schedule where the device needs to be services (e.g., to fix broken components or upgrade certain parts). While at the service center, the device is serviced and can be deployed to the same hospital or a different hospital.

In the service center, the log files are downloaded from the device and uploaded to the device server, and upon successful reception and verification of the log files, the device server approves log file deletion from the device storage. If any boards or components need to be replaced, the device server generates an updated manifest file which is stored on the device.

Part of the log files which are downloaded from the device include a historical record of all messages generated by the device for transmission over LoRaWAN/Wi-Fi since the last service operation. These are also provided on the device server to ensure that no loss of data has occurred.

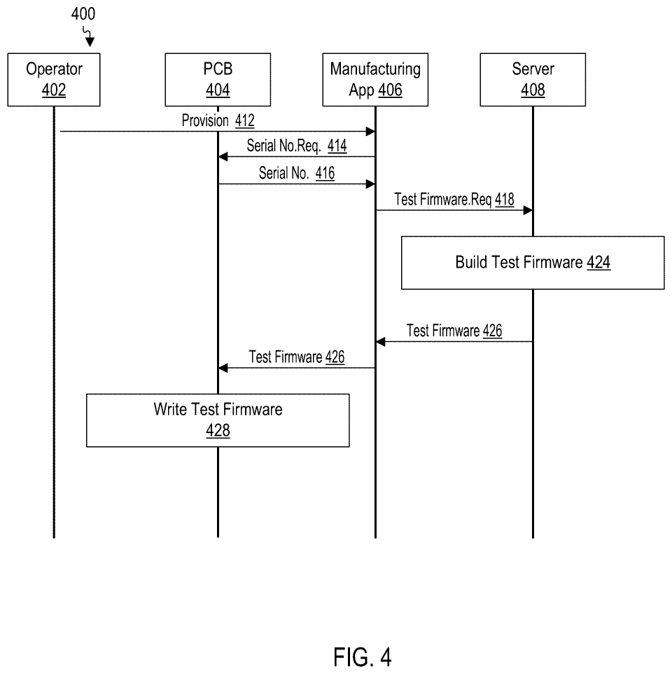

FIG. 4 illustrates a sequence diagram of manufacturing a main printed circuit board (PCB) of a gas delivery device that can be employed in the therapeutic gas delivery system in accordance with aspects of the disclosure. In some cases, the sequence illustrates an operator 402 configuring a manufacturing application 406 (e.g., in the factory/service center) to provision the PCB 404 with information necessary to build the device (e.g., the gas delivery devices 202). In some aspects, the PCB 404 is configured to receive information from a server 408, which builds a test firmware and constructs a manifest to install and build the gas delivery devices 202. Initially, the operator 402 requests provisioning of a new device at the manufacturing application 406, and the manufacturing application 406 sends a serial number request 414 to the PCB 404.

In response, the PCB 404 sends the serial number 416 to the manufacturing application 406. Based on the serial number, the server 408 provides a test firmware request 418 to the server 408, which then builds a test firmware 426 at block 424. In some aspects, the test firmware includes proprietary information such as generated keys that are programmed in the gas delivery devices 202. The server 408 sends the test firmware 426 to the manufacturing application 406, when conveys the test firmware 426 to the PCB 404. At block 428, the PCB 404 writes the test firmware 426 into a non-volatile storage medium, such as a flash memory or EEPROM.

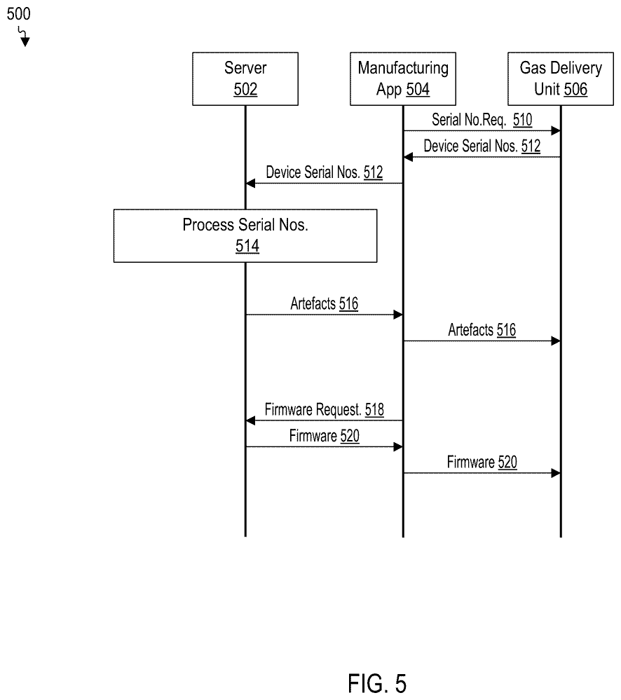

FIG. 5 illustrates a sequence diagram of manufacturing at least a portion of a gas delivery device that can be employed in the therapeutic gas delivery system in accordance with aspects of the disclosure.

In particular, in FIG. 5, a server 502, a manufacturing application 504, and a gas delivery device 506 (e.g., the gas delivery devices 202) are configured to program the various devices with artefacts and other information to enable the communication in a secure manner. In some aspects, the manufacturing application 504 is configured to send a serial number request 510 that requests the serial numbers of the various components of the gas delivery device 506. For example, because the gas delivery device 506 is configured to transmit gas for medical purposes, the gas delivery device 506 may include a number of components such as an actuator to enable the therapeutic gas (e.g., an actuator, a valve, etc.), a wireless communication module, etc. The gas delivery device 506 provides the device serial numbers 512 to the manufacturing application 504 and then conveys the device serial numbers 512 to the server 502. In some examples, the device serial numbers 512 may also include serial numbers or other identifying information for one or more gas sources connected to the gas delivery device.

At block 514, the 506 is configured to process the information and generate various artefacts 516 that will be installed in the gas delivery device 506. The artefacts can include device secret keys binary (populated to the EEPROM of the Main Board), device manifest file, unique device identifier (UDI) file, a device ID file including the device serial number, wireless transmission configuration files and certificate files, a set of permissible product codes for NO cylinders, and language and time Offset settings files.

The artefacts are conveyed to the manufacturing application 504 and then programmed into the gas delivery device 506. At this point, the device is configured for provisioning into production by installing a production firmware. At this point, the manufacturing application 504 sends a firmware request 518 for the latest production firmware from the server 502 and receives the production firmware 520. In some aspects, the dev/ops operation is configured to ensure that the firmware 520 is only provided on secure channels and to known clients using established and future secure techniques. The firmware is then provided from the manufacturing application 504 to the gas delivery device 506 and installed, which overwrites the test firmware. At this point, the gas delivery device 506 has all operational software, but further steps can be taken such as a testing phase, packaging, and so forth.

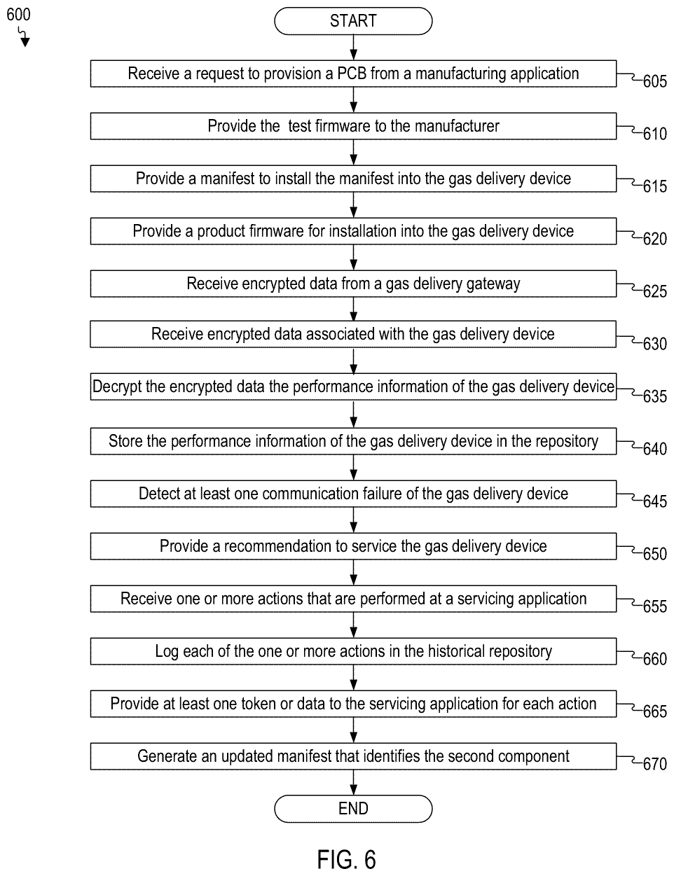

FIG. 6 illustrates an example method 600 of a dev/ops system in accordance with aspects of the disclosure. Although the example method 600 depicts a particular sequence of operations, the sequence may be altered without departing from the scope of the present disclosure. For example, some of the operations depicted may be performed in parallel or in a different sequence that does not materially affect the function of the method 600. In other examples, different components of an example device or system that implements the method 600 may perform functions at substantially the same time or in a specific sequence.

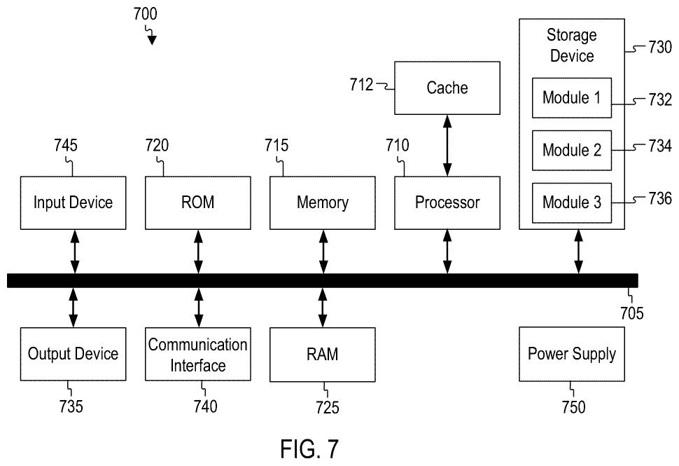

According to some examples, the method includes receiving a request to provision a printed circuit board (PCB) from a manufacturing application executing at a manufacturer at block 605. For example, the processor 710 illustrated in FIG. 7 may receive a request to provision a printed circuit board (PCB) from a manufacturing application executing at a manufacturer. The PCB is integrated into a gas delivery device. The PCB may also provide the serial number to the manufacturing application.

According to some examples, the method includes providing a test firmware to the manufacturer at block 610. For example, the processor 710 illustrated in FIG. 7 may provide the test firmware to the manufacturer. The test firmware is configured to program hardware devices during manufacturing.

According to some examples, the method includes providing a manifest to the manufacturing application to install the manifest into the gas delivery device at block 615. For example, the processor 710 illustrated in FIG. 7 may provide a manifest to the manufacturing application to install the manifest into the gas delivery device. The manifest identifies components that can be replaced in the gas delivery device.

According to some examples, the method includes providing a product firmware to the manufacturing application for installation into the gas delivery device at block 620. For example, the processor 710 illustrated in FIG. 7 may provide a product firmware to the manufacturing application for installation into the gas delivery device.

According to some examples, the method includes receiving encrypted data from a gas delivery gateway that is configured to receive performance information from the gas delivery device at block 625. For example, the processor 710 illustrated in FIG. 7 may receive encrypted data from a gas delivery gateway that is configured to receive performance information from the gas delivery device. The gas delivery device is configured to transmit information to the gas delivery gateway under predetermined circumstances.

According to some examples, the method includes receiving encrypted data from a mobile device that is configured to interface with the gas delivery device at block 630. For example, the processor 710 illustrated in FIG. 7 may receive encrypted data from a mobile device that is configured to interface with the gas delivery device. The gas delivery device is configured to transmit information to the gas delivery gateway under predetermined circumstances. The gas delivery device is configured to identify one of the mobile device or the gas delivery gateway and provide the encrypted data based on whether gas is being delivered. In some aspects, at block 630, when the gas delivery device is not in a shutdown mode and is not delivering the gas, the gas delivery device is configured to transmit the encrypted data on a long-range wireless area network. In some other aspects, at block 630, when the gas delivery device is in a shutdown mode, the gas delivery device is configured to transmit the encrypted data on a long-range wireless area network or a short-range wireless area network. In some aspects, when the gas delivery device is delivering the gas, the gas delivery device is configured to omit transmission of the encrypted data.

According to some examples, the method includes decrypting the encrypted data to yield the performance information and storing the performance information of the gas delivery device in a historical repository at block 635. For example, the processor 710 illustrated in FIG. 7 may decrypt the encrypted data to yield the performance information and storing the performance information of the gas delivery device in a historical repository.

According to some examples, the method includes decrypting the encrypted data to yield the performance information and storing the performance information of the gas delivery device in the historical repository at block 640. For example, the processor 710 illustrated in FIG. 7 may decrypt the encrypted data to yield the performance information and storing the performance information of the gas delivery device in the historical repository.

According to some examples, the method includes detecting at least one communication failure of the gas delivery device at block 645. For example, the processor 710 illustrated in FIG. 7 may detect at least one communication failure of the gas delivery device.

According to some examples, the method includes providing a recommendation to service the gas delivery device at block 650. For example, the processor 710 illustrated in FIG. 7 may provide a recommendation to service the gas delivery device.

According to some examples, the method includes receiving one or more actions that are performed at a servicing application for servicing or analyzing the gas delivery device at block 655. For example, the processor 710 illustrated in FIG. 7 may receive one or more actions that are performed at a servicing application for servicing or analyzing the gas delivery device. The one or more actions comprise an action to replace a first component of the gas delivery device based on an expiration date or a failure.

According to some examples, the method includes logging each of the one or more actions in the historical repository at block 660. For example, the processor 710 illustrated in FIG. 7 may log each of the one or more actions in the historical repository.

According to some examples, the method includes providing at least one token or data to the servicing application for each of the one or more actions at block 665. For example, the processor 710 illustrated in FIG. 7 may provide at least one token or data to the servicing application for each of the one or more actions.

According to some examples, the method includes generating an updated manifest that identifies the second component that replaces the first component at block 670. For example, the processor 710 illustrated in FIG. 7 may generate an updated manifest that identifies the second component that replaces the first component. The action to replace the first component is stored in the historical repository.

FIG. 7 shows an example of computing system 700, which can be for example any computing device making up various parts of the gas delivery system, or any component thereof in which the components of the system are in communication with each other using connection 705. Connection 705 can be a physical connection via a bus, or a direct connection into processor 710, such as in a chipset architecture. Connection 705 can also be a virtual connection, networked connection, or logical connection.

In some embodiments, computing system 700 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 700 includes at least one processing unit (CPU or processor) 710 and connection 705 that couples various system components including system memory 715, such as read-only memory (ROM) 720 and random-access memory (RAM) 725 to processor 710. Computing system 700 can include a cache of high-speed memory 712 connected directly with, in close proximity to, or integrated as part of processor 710.

Processor 710 can include any general purpose processor and a hardware service or software service, such as services 732, 732, and 736 stored in storage device 730, configured to control processor 710 as well as a special-purpose processor where software instructions are incorporated into the actual processor design. Processor 710 may essentially be a completely self-contained computing system, containing multiple cores or processors, a bus, memory controller, cache, etc. A multi-core processor may be symmetric or asymmetric.

To enable user interaction, computing system 700 includes an input device 7 25, which can represent any number of input mechanisms, such as a microphone for speech, a touch-sensitive screen for gesture or graphical input, keyboard, mouse, motion input, speech, etc. Computing system 700 can also include output device 735, which can be one or more of a number of output mechanisms known to those of skill in the art. In some instances, multimodal systems can enable a user to provide multiple types of input/output to communicate with computing system 700. Computing system 700 can include communications interface 7 20, which can generally govern and manage the user input and system output. There is no restriction on operating on any particular hardware arrangement, and therefore the basic features here may easily be substituted for improved hardware or firmware arrangements as they are developed.

Storage device 730 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 730 can include software services, servers, services, etc., that when the code that defines such software is executed by the processor 710, it causes the system to perform a function. In some embodiments, a hardware service that performs a particular function can include the software component stored in a computer-readable medium in connection with the necessary hardware components, such as processor 710, connection 705, output device 735, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Specific details are provided in the description above to provide a thorough understanding of the embodiments and examples provided herein, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Further, those of skill in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Individual embodiments may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

In some embodiments the computer-readable storage devices, mediums, and memories can include a cable or wireless signal containing a bitstream and the like. However, when mentioned, non-transitory computer-readable storage media expressly exclude media such as energy, carrier signals, electromagnetic waves, and signals per se.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof, in some cases depending in part on the particular application, in part on the desired design, in part on the corresponding technology, etc.

The various illustrative logical blocks, modules, and circuits described in connection with the aspects disclosed herein may be implemented or performed using hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods, algorithms, and/or operations described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or

The program code may be executed by a processor, which may include one or more processors, such as one or more DSPs, general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general-purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” or “communicatively coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

As used herein, the terms “medical gas” and “therapeutic gas” may be used interchangeably to mean a gas administered to a patient in need thereof that differs from unmodified breathing gas from a ventilator.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A method of managing a gas delivery device by a server, comprising:
receiving a request to provision a serial number of a printed circuit board (PCB) from a manufacturing application executing at a manufacturer, wherein the PCB is integrated into the gas delivery device;
providing the serial number and a test firmware to the manufacturer, wherein the test firmware is configured to program hardware devices during manufacturing;
providing a product firmware to the manufacturing application for installation into the gas delivery device;
receiving encrypted data from a gas delivery gateway that is configured to receive performance information from the gas delivery device, wherein the gas delivery device is configured to transmit information to the gas delivery gateway under predetermined circumstances;
decrypting the encrypted data to yield the performance information and storing the performance information of the gas delivery device in a historical repository;
receiving one or more actions that are performed at a servicing application for servicing or analyzing the gas delivery device;
logging each of the one or more actions in the historical repository; and
providing at least one token or data to the servicing application for each of the one or more actions.

2. The method of claim 1, further comprising:
receiving the encrypted data from a mobile device that is configured to interface with the gas delivery device, wherein the gas delivery device is configured to transmit information to the gas delivery gateway under predetermined circumstances; and
decrypting the encrypted data to yield the performance information and storing the performance information of the gas delivery device in the historical repository.

3. The method of claim 2, wherein the gas delivery device is configured to identify one of the mobile device or the gas delivery gateway and provide the encrypted data based on whether gas is being delivered.

4. The method of claim 3, wherein, when the gas delivery device is delivering the gas, the gas delivery device is configured to omit transmission of the encrypted data.

5. The method of claim 2, wherein, when the gas delivery device is not in a shutdown mode and is not delivering a gas, the gas delivery device is configured to transmit the encrypted data on a long-range wireless area network.

6. The method of claim 2, wherein, when the gas delivery device is in a shutdown mode, the gas delivery device is configured to transmit the encrypted data on a long-range wireless area network or a short-range wireless area network.

7. The method of claim 1, further comprising:
detecting at least one communication failure of the gas delivery device; and
providing a recommendation to service the gas delivery device.

8. The method of claim 1, further comprising:
providing a manifest to the manufacturing application to install the manifest into the gas delivery device, wherein the manifest identifies components that can be replaced in the gas delivery device.

9. The method of claim 8, wherein the one or more actions comprise an action to replace a first component of the gas delivery device based on an expiration date or a failure.

10. The method of claim 9, further comprising:
generating an updated manifest that identifies a second component that replaces the first component, wherein the action to replace the first component is stored in the historical repository.

11. A system for managing a gas delivery device by a server, the system comprising:
a processor in communication with a memory, the memory including instructions executable by the processor to:
receive a request to provision a serial number of a printed circuit board (PCB) from a manufacturing application executed at a manufacturer, wherein the PCB is integrated into the gas delivery device;
providing the serial number and a test firmware to the manufacturer, wherein the test firmware is configured to program hardware devices during manufacturing;
receive encrypted data from a gas delivery gateway that is configured to receive performance information from the gas delivery device, wherein the gas delivery device is configured to transmit information to the gas delivery gateway under predetermined circumstances;
decrypt the encrypted data to yield the performance information and storing the performance information of the gas delivery device in a historical repository;
receive one or more actions that are performed at a servicing application for servicing or analyzing the gas delivery device;
log each of the one or more actions in the historical repository; and
provide at least one token or data to the servicing application for each of the one or more actions.

12. The system of claim 11, the memory including instructions executable by the processor further configured to:
receive the encrypted data from a mobile device that is configured to interface with the gas delivery device, wherein the gas delivery device is configured to transmit information to the gas delivery gateway under predetermined circumstances; and
decrypt the encrypted data to yield the performance information and storing the performance information of the gas delivery device in the historical repository.

13. The system of claim 12, wherein the gas delivery device is configured to identify one of the mobile device or the gas delivery gateway and provide the encrypted data based on whether gas is being delivered.

14. The system of claim 13, wherein, when the gas delivery device is delivering the gas, the gas delivery device is configured to omit transmission of the encrypted data.

15. The system of claim 12, wherein, when the gas delivery device is not in a shutdown mode and is not delivering a gas, the gas delivery device is configured to transmit the encrypted data on a long-range wireless area network.

16. The system of claim 12, wherein, when the gas delivery device is in a shutdown mode, the gas delivery device is configured to transmit the encrypted data on a long-range wireless area network or a short-range wireless area network.

17. The system of claim 11, the memory including instructions executable by the processor further configured to:
detect at least one communication failure of the gas delivery device; and
provide a recommendation to service the gas delivery device.

18. The system of claim 11, the memory including instructions executable by the processor further configured to provide a manifest to the manufacturing application to install the manifest into the gas delivery device, wherein the manifest identifies components that can be replaced in the gas delivery device.

19. The system of claim 18, wherein the one or more actions comprise an action to replace a first component of the gas delivery device based on an expiration date or a failure.

20. The system of claim 19, the memory including instructions executable by the processor further configured to generate an updated manifest that identifies a second component that replaces the first component, wherein the action to replace the first component is stored in the historical repository.
